(19) United States
(12) Patent Application Publication
(10) Pub. No.: US 2022/0005598 A1
(43) Pub. Date: Jan. 6, 2022

(54) DYNAMIC SCHEDULING OF THE DISTRIBUTION OF MEDICAL EQUIPMENT

(71) Applicant: Inlecom Systems Limited, London (GB)

(72) Inventors: Ibad Kureshi, Spennymoor (GB); Jenny Rainbird, Bexleyheath (GB); Ioanna Fergadiotou, Ntrafi-Pikermi (GR); Antonios Mygiakis, Chalandri (GR); Patrick J. O'Sullivan, Dublin (IE)

(21) Appl. No.: 16/920,214

(22) Filed: Jul. 2, 2020

(51) Int. Cl. G16H 40/40; G16H 40/20; G06Q 10/06

(52) U.S. Cl. CPC G16H 40/40; G06Q 10/06314; G16H 40/20

(57) ABSTRACT

The dynamic scheduling of medical equipment distribution includes loading a table correlating different medical devices with correspondingly assigned patients contemporaneously utilizing the devices, and correspondingly assigned patients scheduled for subsequent utilization of the devices. One device is geolocated at a drop off location at a date prior to a date indicated in the table when the device is to be returned. Thereafter, a patient is identified who is scheduled for subsequent utilization of another device at a specific date past the date prior. A message is then transmitted to the identified patient specifying an early pickup opportunity for the geolocated device at the drop off location before the specific date. In response to an affirmation from the identified patient, a record in the table is modified assigning the identified patient to pick up the geolocated device at the drop off location and the other device reassigned to a different patient.

### BACKGROUND OF THE INVENTION

### Field of the Invention

The present invention relates to the field of medical device utilization and more particularly to the distribution of re-usable medical devices over a disperse geographic area to different patients utilizing the devices and the re-distribution of the devices to other patients.

### Description of the Related Art

In the field of health care delivery, medical devices, particularly diagnostic devices and devices used to collect biological samples, automate the delivery of medicine or to manipulate anatomical portions of the body, have become an integral component of modern medicine. In many instances, medical devices are no more complex than a container to hold a fluid or tissue sample, or a syringe adapted to deliver a drug to a patient. In other instances, medical devices are large, immovable machines operable to image portions of the anatomy or to delivery life saving, life preserving support and life preserving medicines. But, in many instances, complex devices—re-usable devices—are small enough to be portable and thus used by a patient away from a central point of care such as a hospital or physician's office.

In respect to portability and re-usable medical devices, generally these devices are small enough to be transported by an individual by way of a personal vehicle. As well, these devices in all cases are expensive enough to require repeated use by different patients without permitting a one-time use and disposal of the device by a single patient. Examples include portable oxygen delivery systems, mobility aids such as crutches, canes or electric scooters, portable dialysis units, breast pumps, continuous positive airway pressure machines, and portable ultrasound diagnostic imaging devices, to name only a few examples. To the extent that a health care delivery organization has in their inventory a discrete number of portable devices, but very many prospective patients in need of those devices, an efficient way of scheduling use by each of the patients is required.

While coordinating a schedule of use by different patients of a device is a task simple enough, doing so across a disperse geographic area where the devices are pre-positioned at different locations is more of a challenge. In particular, it is desirable to schedule the allocation and the retrieval of a device by a patient at a location nearest the patient so as to minimize the time and resources consumed relative to the patient needing the device from a contemporaneous location. But, in many instances, too few devices are available for pickup at one location and too many at another. As well, even when an optimal number of devices reside at a particular location, the assignment of those devices to different patients only accounts for notions of “first come, first served” without consideration for the urgency of access to a device associated with particular patients.

General solutions today include only enhancing inventory of the devices at the locations in greatest need, which can have significant cost implications as such devices are known to be very expensive. Other solutions include extending the time during which a patient must wait to retrieve the needed device or requiring the patient to travel a greater distance to a further pick up location in order to retrieve a needed device. In either instance, utilization and wastage inefficiencies are present owing to delays in patients having a greatest medical need for the use of a device whilst the device is used by a patient of lesser medical need. Consequently, none of the foregoing approaches are optimal.

### BRIEF SUMMARY OF THE INVENTION

Embodiments of the present invention address deficiencies of the art in respect to medical device distribution and re-distribution amongst end users and provide a novel and non-obvious method, system and computer program product for the dynamic scheduling of the distribution of medical equipment. In an embodiment of the invention, a method for dynamically scheduling the distribution of medical equipment includes loading into memory of a computer, a table correlating different medical devices, and both correspondingly assigned patients utilizing the devices and corresponding assigned patients scheduled for subsequent utilization of the devices, as well as a geographic locations to which ones of the medical devices are to be returned by the correspondingly assigned patients utilizing the devices, and from which ones of the medical devices are to be retrieved by the correspondingly assigned patients scheduled for subsequent utilization of the devices and a date and time for each of the devices by which each of the devices is to be returned to a corresponding one of the geographic locations.

The method additionally includes geolocating one of the devices at a drop off location and determining from the table, that the geolocated one of the devices has been returned to the drop off location at a date prior to a date indicated in the table when the geolocated one of the devices had been expected to be returned at the drop off location. The method yet further includes identifying one of the patients scheduled for subsequent utilization of another of the devices at a specific date past the date prior. The method yet further includes transmitting a message to the identified one of the patients specifying a pickup opportunity for the geolocated one of the devices at the drop off location before the specific date and, in response to an affirmation from the identified one of the patients, modifying a record in the table assigning the identified one of the patients to pick up the geolocated one of the devices at the drop off location and reassigning the another of the devices at the pickup location to a different one of the patients.

In one aspect of the embodiment, the method additionally includes detecting a utilization of the one of the devices wirelessly from over a computer communications network prior to geolocating the one of the devices at the drop off location, determining based upon the detected utilization that the one of the devices is likely to be returned to the drop off location at the date prior, and transmitting the message to the identified one of the patients in response to the detection and before the one of the devices is geolocated at the drop off location. In another aspect of the embodiment, the method additionally includes reading accelerometer data for the one of the devices and suppressing transmission of the message on condition that the data indicates a dropping of the one of the devices indicating a maintenance requirement for the one of the devices. In yet another aspect of the embodiment, the method additionally includes initiating a timer in respect to the one of the devices and suppressing transmission of the message on condition that the timer has lapsed indicating a service cycle requirement for the one of the devices.

In a further aspect of the embodiment, the method additionally includes identifying a set of the patients scheduled for subsequent utilization of another of the devices at respectively different pickup locations that each differ from the drop off location at a specific date, retrieving an indication of urgency for each of the patients in the set and transmitting the message to a selected one the patients in the set having a highest indication of urgency. In yet further an aspect of the embodiment, the method additionally includes identifying a set of the patients scheduled for subsequent utilization of another of the devices at the drop off location, each at a specific date past the date prior, retrieving an indication of urgency for each of the patients in the set and transmitting the message to a selected one the patients in the set having a highest indication of urgency.

In another embodiment of the invention, a location-based data processing system is configured for dynamically scheduling the distribution of medical equipment. The system includes a host computing platform with one or more computers, each with memory and at least one processor. The system also includes fixed storage coupled to the host computing platform. The fixed storage stores a table that correlates different medical devices with both correspondingly assigned patients utilizing the devices and corresponding assigned patients scheduled for subsequent utilization of the devices as well as a geographic locations to which ones of the medical devices are to be returned by the correspondingly assigned patients utilizing the devices, and from which ones of the medical devices are to be retrieved by the correspondingly assigned patients scheduled for subsequent utilization of the devices and a date and time for each of the devices by which each of the devices is to be returned to a corresponding one of the geographic locations. Finally, the system includes a dynamic scheduling module executing in the memory of the host computing platform.

The module includes computer program instructions causing the at least one processor to load the table into the memory of the host computing platform, geolocate one of the devices at a drop off location and determine from the table, that the geolocated one of the devices has been returned to the drop off location at a date prior to a date indicated in the table when the geolocated one of the devices had been expected to be returned at the drop off location. The program instructions then identify one of the patients scheduled for subsequent utilization of another of the devices at a specific date past the date prior. The computer program instructions additionally cause the at least one processor to transmit a message to the identified one of the patients specifying a pickup opportunity for the geolocated one of the devices at the drop off location before the specific date and, in response to an affirmation from the identified one of the patients, to both modify a record in the table assigning the identified one of the patients to pick up the geolocated one of the devices at the drop off location and while also reassigning the another of the devices at the pickup location to a different one of the patients.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is pictorial illustration of a process for dynamically scheduling the distribution of medical equipment;

FIG. 2 is a schematic diagram of a location-based data processing system configured for dynamically scheduling the distribution of medical equipment; and,

FIG. 3 is a flow chart illustrating a process for dynamically scheduling the distribution of medical equipment.

### DETAILED DESCRIPTION OF THE INVENTION

Embodiments of the invention provide for the dynamic scheduling of the distribution of medical equipment across a disperse geographic area. In accordance with an embodiment of the invention, a table is maintained correlating individual portable medical devices with each of a correspondingly assigned patient contemporaneously utilizing the device, a patient scheduled to receive the device for subsequent utilization after the assigned patient, and a drop off location at which the device is to be returned by the assigned patient and at which the device is to be retrieved by the patient scheduled to receive the device for subsequent utilization, and a timeframe when the device is scheduled to be returned by the assigned patient and retrieved by a next patient. Thereafter, a selected one of the devices may be geolocated at a respective drop off location prior to a date when the selected device is expected to be present at the drop off location indicating an early return of the device.

In response, the table is consulted to identify a corresponding patient who had been scheduled for subsequent utilization of a different device at a later date intermediate to the date when the selected one of the devices had been geolocated, and a date when the corresponding patient had been scheduled to retrieve the different device. A message is then transmitted to the identified patient specifying the geolocated device at the drop off location and requesting that the identified patient retrieve the geolocated device at the drop off location in lieu of the different device. In response to an affirmation from the identified patient, a record in the table is modified assigning the identified patient to pick up the geolocated device at the drop off location and the different device is reassigned for subsequent retrieval by yet a different patient. In this way, the re-utilization of the device may be optimized so as to shorten the wait time by the identified patient in receiving use of the required device. Further, when applied to all of the devices dispersed within the geographic area, the total utilization of the devices per patient can be increased owing to the increased optimization of assignment of subsequent utilization of each of the devices.

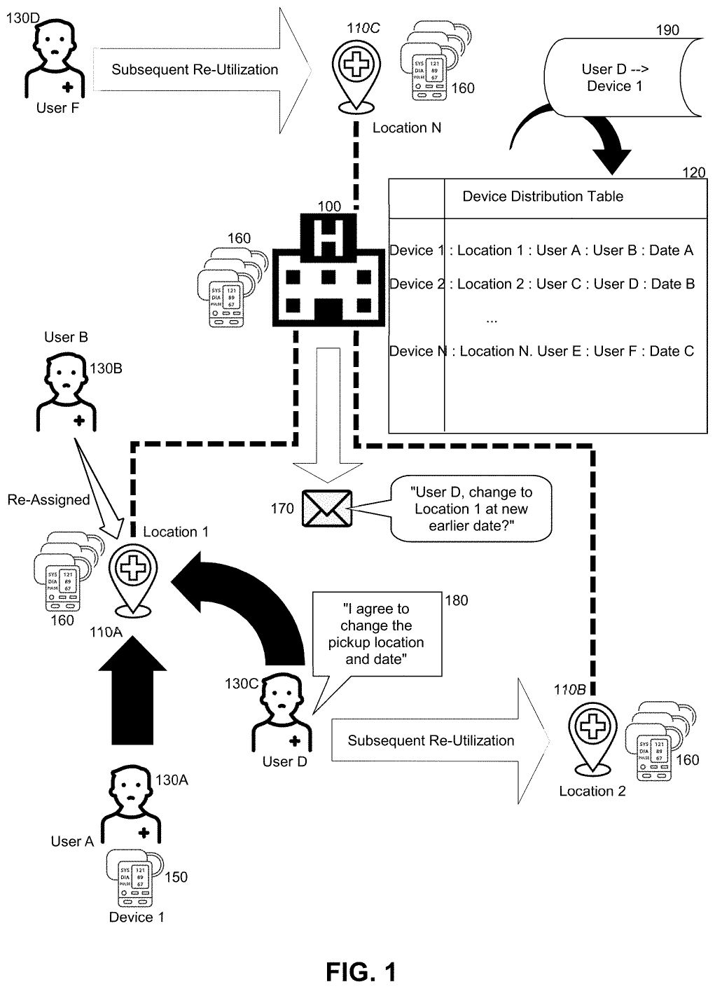

In further illustration, FIG. 1 pictorially shows a process for dynamically scheduling the distribution of medical equipment. As shown in FIG. 1, different units 160 of a portable medical device are distributed to distribution points 110A, 110B, 110C about a geographic area from a central location 100, the distribution points 110A, 110B, 110C including, by way of example, different healthcare facilities such as doctor's offices, public healthcare units and medical supply storefronts. A table 120 is then maintained correlating each corresponding one of the different units 160 with a specific one of the distribution points 110A, 110B, 110C from which the corresponding one of the units 160 had most recently been distributed to a contemporaneously utilizing one of different patients 130A. The table 120 further correlates each of the different units 160 with a scheduled one of the different patients 130B, 130C, 130D designated to receive the corresponding one of the units 160 for re-utilization once of the corresponding one of the units 160 has been returned to the specific one of the distribution points 110A, 110B, 110C by the contemporaneously utilizing one of the different patients 130A. Of note, any one of the units 160 may be returned one of the distribution points 110A, 110B, 110C that differs from another of the distribution points 110A, 110B, 110C from which the one of the units 160 had previously been retrieved.

Optionally, the table 120 also specifies for each of the units 160, a timeframe by which the corresponding one of the units 160 is to be returned by the contemporaneously utilizing one of the different patients 130A to the specific one of the distribution points 110A, 110B, 110C. As well, the table 120 can include, for each of the units 160, a timeframe by when the scheduled one of the different patients 130B, 130C, 130D is to retrieve the corresponding one of the units 160 for a specific one of the distribution points 110A, 110B, 110C. Even further, the table 120 can include, for each of the units 160, an indication of whether or not a corresponding one of the units 160 has been utilized by the contemporaneously utilizing one of the different patients 130A. Even yet further, the table 120 can include, for each of the units 160, an indication of whether or not the corresponding one of the units 160 requires maintenance based upon the utilization of the corresponding one of the units 160 by the contemporaneously one of the utilizing one of the different patients 130A.

Of note, the utilizing one of the different patients 130A may return a contemporaneously utilized unit 150 from amongst the units 160 to a location 110A at a time and date before the timeframe indicated in the table 120 for the contemporaneously utilized unit 150 and before the timeframe in the table 120 indicates when a next scheduled one of the different patients 130B is to receive the contemporaneously utilized unit 150 at the location 110A. In this instance, the table 120 may be queried to identify one or more others of the patients 130C, 130D scheduled to receive a corresponding one of the units 160 at the location 110A, or at different locations 110B, 110C and on a date that is intermediate to when the contemporaneously utilized unit 150 has arrived at the location 110A and the time according to the timeframe of the contemporaneously utilized unit 150 when the next scheduled one of the different patients 130B is to receive the contemporaneously utilized unit 150.

One of the patients 130C, 130D may then be selected to receive the contemporaneously utilized unit 150 at the location 110A instead of waiting to receive another of the units 160 at the location 110A, or at one of the different location 110B, 110C. In particular, one of the patients 130C, 130D determined to have the highest degree of medical urgency indicated within the table 120 may be selected to receive the contemporaneously utilized unit 150. Alternatively, the one of the patients 130C, 130D may be selected on a first-come, first-served basis in that a first one of the patients 130C, 130D to respond to a message 170 offering a change in scheduling to retrieve the contemporaneously utilized unit 150 now returned at the location 110A may be selected to receive the contemporaneously utilized unit 150 at the location 110A.

To that end, the selected patient or patients 130C, 130D may receive an electronic message 170 prompting to accept a change in location and timeframe for receiving one of the units 160. The message 170 may be transmitted upon the contemporaneously utilized unit 150 arriving at the location 110A, or beforehand upon receiving a message from the contemporaneously utilized unit 150 from over a computer communications network indicating a utilization of the unit 150 by the utilizing one of the different patients 130A thus predicting an imminent return of the unit 150 to the location 110A. However, the transmission of the message 170 may be suppressed in the event that accelerometer data is provided in the message received from the unit 150 indicative of a dropping of the unit 150 and a need for maintenance and repair of the unit 150, or the lapsing of a timer for the unit 150 indicating a need for routine maintenance of the unit 150.

Upon receiving an affirmance 180 from a first one of the selected patients 130C, 130D or the selected patient 130C, a modification 190 is applied to the record in the table 120 corresponding to the contemporaneously utilized unit 150 replacing the next scheduled one of the different patients 130B with the selected patient 130C and the next scheduled one of the different patients 130B is then assigned to a different one of the units 160 within the table 120. In this way, the scheduling of the distribution of the unit 150 is optimized according to the timing of return of the unit 150 to the location 110A and the needs of the other patients 130C, 130D awaiting re-utilization of a different one of the units 160 from other locations 110B, 110C.

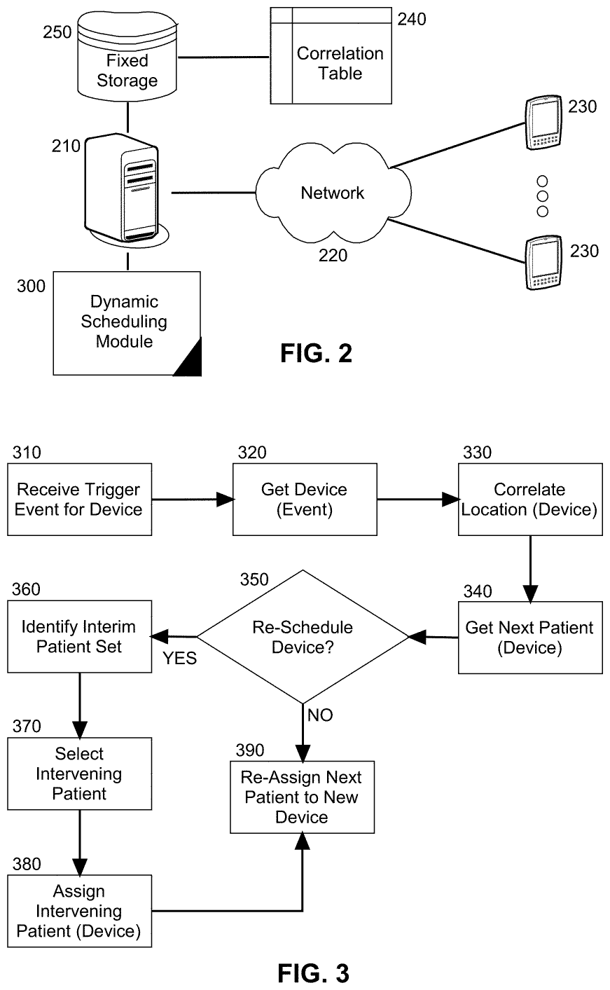

The process described in connection with FIG. 1 may be implemented within a location-based data processing system. In further illustration, FIG. 2 schematically shows a location-based data processing system configured for dynamically scheduling the distribution of medical equipment. The system includes a host computing platform 210 that includes at least one computer with memory and at least one processor. Fixed storage 250 is coupled to the host computing platform 210 and stores therein a correlation table 240. The correlation table 240 stores a set of records, the records individually or in combination with other records, correlating different medical device units with a specific distribution point from which a corresponding one of the units had most recently been distributed to a contemporaneously utilizing patients. The table 240 further correlates each unit with a scheduled patient designated to receive a corresponding one of the units for re-utilization once the corresponding unit has been returned to the specific distribution points by the contemporaneously utilizing patient.

Optionally, the table 240 also specifies for each unit a timeframe by which the unit is to be returned by the contemporaneously utilizing patient to the specific distribution point. As well, the table 240 can include, for each unit, a timeframe by when the scheduled patient is to retrieve the unit from the specific distribution point. Even further, the table 240 can include, for each unit, an indication of whether or not the unit has been utilized by the contemporaneously utilizing one of the different patients. Even yet further, the table 240 can include, for each unit, an indication of whether or not the unit requires maintenance based upon the utilization of the unit by the utilizing patient.

Of note, the system includes a dynamic scheduling module 300. The module 300 includes computer program instructions that when executing in the memory of the host computing platform, are enabled to detect the return of a unit to a corresponding location and to query the table 240 with the unit in order to identify a next patient scheduled to retrieve the unit and a timeframe when the next patient is to retrieve the unit from the location. On the condition that the unit has been returned to the location before the timeframe, the program instructions further query the table 240 to identify other patients already scheduled to retrieve other units from the same location or from other locations on a date that is intermediate to a contemporaneous date of the return of the unit to the location, and the timeframe of the returned unit.

The program instructions then select one of the patients in a result set to the query and transmit a message over computer communications network 220 to a computing device 230 of the selected one of the patients prompting the selected one of the patients to accept an earlier retrieval of the device at a location that differs from the originally scheduled location. Upon receiving an affirming response from the mobile device 230 from over the computer communications network, the program instructions modify the table 240 to specify the selected patient as the next patient to retrieve the unit from the location and to specify the originally scheduled next patient to retrieve either a different unit at the same or a different location, or to simply change the timeframe of retrieving the unit to accommodate the utilization of the unit by the selected patient.

In even further illustration of the operation of the dynamic scheduling module in one aspect of the embodiment of FIG. 2, FIG. 3 is a flow chart illustrating a process for dynamically scheduling the distribution of medical equipment. Beginning in block 310, a trigger event is received from a medical device unit. For instance, the triggering event may be the detected arrival of the unit at a distribution point, the utilization of the unit by a contemporaneously utilizing patient, the lapsing of a maintenance timer for the unit indicating a need to subject the unit to servicing and maintenance, or accelerometer information indicating a dropping of the unit and a need for repairing of the unit. In block 320, a unit corresponding to the event is identified and in block 330, the unit is correlated to a location at which the unit is to be re-utilized by a next scheduled patient. In block 340, the next scheduled patient is then retrieved from a database in correspondence to the location and the unit.

In decision block 350, it is determined if the unit is determined to prohibit rescheduling, owing to a need for maintenance or repair, the next scheduled patient is rescheduled to retrieve a different unit at the same or a different location in block 390. However, if in decision block 350 it is determined that the unit permits rescheduling, in block 360, an interim set patients is determined each of whom are scheduled to re-utilize different units at different locations, but not at the location when the unit has arrived or is anticipated to arrive. In block 370, one of the patients is selected, for instance a patient known to have the greatest urgency of use of the unit, or a patient first responding to a query to retrieve the unit. Then, in block 380, the selected patient is assigned to retrieve the unit at the location whilst, in block 390, the next scheduled patient is rescheduled to retrieve a different unit at the same or different location, or to retrieve the same unit albeit according a timeframe accounting for the utilization of the unit by the selected patient.

As can be seen, the foregoing process one to view in the aggregate, an optimization from a planned distribution of multiple units to different patients over an expansive time, to an actual distribution of the units to the different patients over a compressed time. Specifically, by capitalizing on the dynamic rescheduling of the distribution of the units to the patients in response to the early return of some of the units, more patients receive use of the same number of units in a shorter period of time. This optimization permits the removal of some of the units from distribution to accommodate maintenance and repair without affecting the planned distribution of units to the patients by a time and date certain.

Notably, by observing the difference between the planned distribution and the actual distribution, future adjustments in inventory levels can be intelligently determined by assessing how many units must remain in inventory at each distribution point to accommodate a desired distribution of the units to a select number of patients within a specific timeframe. More to the point, whereas one inventory level of units may be assumed to service a set number of patients from one distribution point, the optimization of distribution afforded by the foregoing process permits a reduced inventory level of units to accommodate the same number of patients from the distribution point within the same time timeframe.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein includes an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which includes one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. A method for dynamically scheduling the distribution of medical equipment, the method comprising:
loading into memory of a computer, a table correlating different medical devices with both correspondingly assigned patients utilizing the devices and correspondingly assigned patients scheduled for subsequent utilization of the devices as well as a geographic locations to which ones of the medical devices are to be returned by the correspondingly assigned patients utilizing the devices, and from which ones of the medical devices are to be retrieved by the correspondingly assigned patients scheduled for subsequent utilization of the devices and a date and time for each of the devices by which each of the devices is to be returned to a corresponding one of the geographic locations;
geolocating one of the devices at a drop off location and determining from the table, that the geolocated one of the devices has been returned to the drop off location at a date prior to a date indicated in the table when the geolocated one of the devices had been expected to be returned at the drop off location;
identifying one of the patients scheduled for subsequent utilization of another of the devices at a specific date past the date prior;
transmitting a message to the identified one of the patients specifying a pickup opportunity for the geolocated one of the devices at the drop off location before the specific date; and,
responsive to an affirmation from the identified one of the patients, modifying a record in the table assigning the identified one of the patients to pick up the geolocated one of the devices at the drop off location and reassigning the another of the devices at the pickup location to a different one of the patients.

2. The method of claim 1, further comprising:
detecting a utilization of the one of the devices wirelessly from over a computer communications network prior to geolocating the one of the devices at the drop off location;
determining based upon the detected utilization that the one of the devices is likely to be returned to the drop off location at the date prior; and,
transmitting the message to the identified one of the patients in response to the detection and before the one of the devices is geolocated at the drop off location.

3. The method of claim 1, further comprising:
reading accelerometer data for the one of the devices; and,
suppressing transmission of the message on condition that the data indicates a dropping of the one of the devices indicating a maintenance requirement for the one of the devices.

4. The method of claim 1, further comprising:
initiating a timer in respect to the one of the devices; and,
suppressing transmission of the message on condition that the timer has lapsed indicating a service cycle requirement for the one of the devices.

5. The method of claim 1, further comprising:
identifying a set of the patients scheduled for subsequent utilization of another of the devices at respectively different pickup locations that each differ from the drop off location at a specific date;
retrieving an indication of urgency for each of the patients in the set; and,
transmitting the message to a selected one the patients in the set having a highest indication of urgency.

6. The method of claim 1, further comprising:
identifying a set of the patients scheduled for subsequent utilization of another of the devices at the drop off location, each at a specific date past the date prior;
retrieving an indication of urgency for each of the patients in the set; and,
transmitting the message to a selected one the patients in the set having a highest indication of urgency.

7. A location-based data processing system configured for dynamically scheduling the distribution of medical equipment, the system comprising:
a host computing platform comprising one or more computers, each with memory and at least one processor;
fixed storage coupled to the host computing platform, the fixed storage storing a table correlating different medical devices with both correspondingly assigned patients utilizing the devices and correspondingly assigned patients scheduled for subsequent utilization of the devices as well as a geographic locations to which ones of the medical devices are to be returned by the correspondingly assigned patients utilizing the devices, and from which ones of the medical devices are to be retrieved by the correspondingly assigned patients scheduled for subsequent utilization of the devices and a date and time for each of the devices by which each of the devices is to be returned to a corresponding one of the geographic locations; and,
a dynamic scheduling module executing in the memory of the host computing platform, the module comprising computer program instructions causing the at least one processor to perform:
loading the table into the memory of the host computing platform;
geolocating one of the devices at a drop off location and determining from the table, that the geolocated one of the devices has been returned to the drop off location at a date prior to a date indicated in the table when the geolocated one of the devices had been expected to be returned at the drop off location;
identifying one of the patients scheduled for subsequent utilization of another of the devices at a specific date past the date prior;
transmitting a message to the identified one of the patients specifying a pickup opportunity for the geolocated one of the devices at the drop off location before the specific date; and,
responsive to an affirmation from the identified one of the patients, modifying a record in the table assigning the identified one of the patients to pick up the geolocated one of the devices at the drop off location and reassigning the another of the devices at the pickup location to a different one of the patients.

8. The system of claim 7, wherein the program instructions further perform:
detecting a utilization of the one of the devices wirelessly from over a computer communications network prior to geolocating the one of the devices at the drop off location;
determining based upon the detected utilization that the one of the devices is likely to be returned to the drop off location at the date prior; and,
transmitting the message to the identified one of the patients in response to the detection and before the one of the devices is geolocated at the drop off location.

9. The system of claim 7, wherein the program instructions further perform:
reading accelerometer data for the one of the devices; and,
suppressing transmission of the message on condition that the data indicates a dropping of the one of the devices indicating a maintenance requirement for the one of the devices.

10. The system of claim 7, wherein the program instructions further perform:
initiating a timer in respect to the one of the devices; and,
suppressing transmission of the message on condition that the timer has lapsed indicating a service cycle requirement for the one of the devices.

11. The system of claim 7, wherein the program instructions further perform:
identifying a set of the patients scheduled for subsequent utilization of another of the devices at respectively different pickup locations that each differ from the drop off location at a specific date;
retrieving an indication of urgency for each of the patients in the set; and,
transmitting the message to a selected one the patients in the set having a highest indication of urgency.

12. The system of claim 7, wherein the program instructions further perform:
identifying a set of the patients scheduled for subsequent utilization of another of the devices at the drop off location, each at a specific date past the date prior;
retrieving an indication of urgency for each of the patients in the set; and,
transmitting the message to a selected one the patients in the set having a highest indication of urgency.

13. A computer program product for dynamically scheduling the distribution of medical equipment, the computer program product including a computer readable storage medium having program instructions embodied therewith, the program instructions executable by a device to cause the device to perform a method including:
loading into memory of a computer, a table correlating different medical devices with both correspondingly assigned patients utilizing the devices and correspondingly assigned patients scheduled for subsequent utilization of the devices as well as a geographic locations to which ones of the medical devices are to be returned by the correspondingly assigned patients utilizing the devices, and from which ones of the medical devices are to be retrieved by the correspondingly assigned patients scheduled for subsequent utilization of the devices and a date and time for each of the devices by which each of the devices is to be returned to a corresponding one of the geographic locations;
geolocating one of the devices at a drop off location and determining from the table, that the geolocated one of the devices has been returned to the drop off location at a date prior to a date indicated in the table when the geolocated one of the devices had been expected to be returned at the drop off location;
identifying one of the patients scheduled for subsequent utilization of another of the devices at a specific date past the date prior;
transmitting a message to the identified one of the patients specifying a pickup opportunity for the geolocated one of the devices at the drop off location before the specific date; and,
responsive to an affirmation from the identified one of the patients, modifying a record in the table assigning the identified one of the patients to pick up the geolocated one of the devices at the drop off location and reassigning the another of the devices at the pickup location to a different one of the patients.

14. The computer program product of claim 13, wherein the method further includes:
detecting a utilization of the one of the devices wirelessly from over a computer communications network prior to geolocating the one of the devices at the drop off location;
determining based upon the detected utilization that the one of the devices is likely to be returned to the drop off location at the date prior; and,
transmitting the message to the identified one of the patients in response to the detection and before the one of the devices is geolocated at the drop off location.

15. The computer program product of claim 13, wherein the method further includes:
reading accelerometer data for the one of the devices; and,
suppressing transmission of the message on condition that the data indicates a dropping of the one of the devices indicating a maintenance requirement for the one of the devices.

16. The computer program product of claim 13, wherein the method further includes:
initiating a timer in respect to the one of the devices; and,
suppressing transmission of the message on condition that the timer has lapsed indicating a service cycle requirement for the one of the devices.

17. The computer program product of claim 13, wherein the method further includes:
identifying a set of the patients scheduled for subsequent utilization of another of the devices at respectively different pickup locations that each differ from the drop off location at a specific date;
retrieving an indication of urgency for each of the patients in the set; and,
transmitting the message to a selected one the patients in the set having a highest indication of urgency.

18. The computer program product of claim 13, wherein the method further includes:
identifying a set of the patients scheduled for subsequent utilization of another of the devices at the drop off location, each at a specific date past the date prior;
retrieving an indication of urgency for each of the patients in the set; and,
transmitting the message to a selected one the patients in the set having a highest indication of urgency.
